(19) United States
(12) Patent Application Publication
(10) Pub. No.: US 2012/0225127 A1
(43) Pub. Date: Sep. 6, 2012

(54) CLAY NANOCOMPOSITE FORMING MICROCAPSULE USEFUL FOR GUEST ENCAPSULATION AND PROCESS THEREOF

(75) Inventors: Chorappan Pavithran, Thiruvananthapuram (IN); Bindu Prasannakumaran Nair, Thiruvananthapuram (IN)

(73) Assignee: Council of Scientific & Industrial Research, New Delhi (IN)

(21) Appl. No.: 13/320,229

(22) PCT Filed: Mar. 29, 2010
(86) PCT No.: PCT/IN10/00200

§ 371 Date: Apr. 20, 2012

(30) Foreign Application Priority Data
May 12, 2009 (IN) 969/DEL/2009

(51) Int. Cl. A61K 47/34; A61K 9/50; C08K 5/549; B82Y 5/00

(52) U.S. Cl. 424/497; 524/188; 523/209; 514/772.1; 977/788; 977/906

(57) ABSTRACT

A nanocomposite exhibiting solvent-assisted self-assemblage properties and forming microcapsule useful for guest-encapsulation and process for making the same which comprises the reaction product of a smectite-type clay and an oligosilsesquioxane from hydrolytic polycondensation of a trialkoxy aminoalkyl silane and a trialkoxy alkenyl silane dispensed in a vinyl polymer by in situ intercalative polymerisation of a vinyl monomer. A process for making microcapsule which comprises casting a solution of the nanocomposite in a suitable volatile solvent followed by evaporation of the solvent. A method for producing guest-encapsulated microcapsule which comprises casting a solution of the nanocomposite and the guest molecule in a suitable volatile solvent followed by evaporation of the solvent.

### CROSS REFERENCE TO RELATED APPLICATIONS

This application is a National Stage filing under 35 U.S.C. §371 of PCT Application No. PCT/IN2010/000200, filed Mar. 29, 2010. This application also claims the benefit of Indian Patent Application No. 969/DEL/2009, filed May 12, 2009. The content of both applications is incorporated herein by reference in its entirety.

### BACKGROUND OF THE INVENTION

The present invention relates to a nanocomposite forming microcapsule useful for guest encapsulation and process thereof. More particularly, the present invention relates to a polymer-clay nanocomposite exhibiting solvent-assisted self-assemblage properties and forming microcapsule. The nanocomposite microcapsule can find applications such as micro-storage system and encapsulation/delivery of cosmetically active molecules, dyes, catalysts etc.

In response to the growing need for encapsulation materials, several different routes to hollow polymer and ceramic nano and microcapsules have been attempted. For instances, amphiphilic block copolymers, rod-coil diblock copolymers, dendrimers, and amphiphilic fullerene derivatives, see for examples, Antonietti et al, Adv. Mater. 2003, 15, 1323; Discher et al, U.S. Pat. No. 7,217,427; Prud'homme et al, U.S. Pat. No. 7,151,077, were shown to form nano or micro capsules by self-assemblage in selective solvents. In a slight variation from the above, organic-inorganic hybrid capsules, see for examples, Feldheim et al, U.S. Pat. No. 6,602,932; Schmidt et al, J. Am. Chem. Soc. 2003, 125, 14710, have been prepared from organic-inorganic hybrid block polymers.

Ceramic capsules with porous membrane provide encapsulation materials with superior stability and resistance to many external stimuli when compared to encapsulation materials from an organic polymer. But they often need a tedious synthetic strategy involving layer-by-layer coatings of ceramic precursors on a sacrificial template and removal of the template, see for example, Caruso et al, Chem. Mater., 2001, 13, 400.

It is well known that clays, also known as layered-silicates which are constituted from aluminosilicate layers of thickness of about 1 nm and lateral dimensions in the range of several nanometers, when dispersed in polymers produce polymer-clay nanocomposites, also termed as polymer/layered-silicate nanocomposites, which often exhibit remarkable improvement in mechanical, thermal and barrier properties when compared with the virgin polymer. Various polymer/clay nanocomposite systems and their processes, structure and properties are described in published literature, see for example, Ray et al, Prog. Polym. Sci, 2003, 28,1539; Tjong et al, Mater. Sci. Eng R. 2006, 53,73; Pinnavaia et al, U.S. Pat. No. 5,866,645; Lan et al, U.S. Pat. No. 6,387,996; Robello et al, U.S. Pat. No. 6,867,255.

In general, polymer-clay nanocomposites were prepared by three different methods, viz.; 1) Intercalation of polymer by dispersing the clay in a polymer solution (2) In situ intercalative polymerization method which involves swelling of the clay is a liquid monomer followed by polymerization of the monomer and (3) Melt intercalation method which involves annealing, statically or under shear, a mixture of polymer and clay above the softening point of the polymer.

For preparation of polymer-clay nanocomposites, the hydrophilic clays are often organo-modified by exchange of inorganic cations, such as Na⁺ in the clay with organic cations such as alkyl substituted ammonium, imidazolium, phosphonium, pyridinium, and iminium. Recently, organoclays have also been prepared by using silsesquioxanes, particularly polyhedral oligomeric silsesquioxane octamers, having general formula of (RSiO_{1.5})₈ where R represents organic functional groups, as organo-modifier having high thermal stability, see for examples, Fox et al, Langmuir 2007, 23, 7707; Liu et al, Polymer 2005, 46, 157.

In general, polymer-clay compositions form three different types of composites, namely (1) intercalated nanocomposites, for which intercalation of polymer chains into the layered silicate structure occurs in a crystallographically regular fashion (2) flocculated nanocomposites, for which intercalated and stacked silicate layers flocculate to some extent due to the hydroxylated edge-edge interactions of the silicate layers, and (3) exfoliated nanocomposites, for which the individual silicate layers are separated in the polymer matrix by average distances that depend on the clay loading. Polymer-clay nanocomposites with radically different structures such as 3D network, hexagonally patterned lamellar structure, silicate armored polymer particle have also been reported. (Haraguchi et al Macromolecules, 2005, 38, 3482., Toombes et al, Chem. Mater., 2008, 20, 3278., Herrera et al, Macromol. Rapid Commun. 2007, 28, 1567).

Despite the disclosure of the foregoing methods, no reports are there which explored the possibility of synthesising polymer-clay nanocomposite for guest encapsulation. In the present invention, polymer-clay nanocomposite exhibiting solvent- assisted self-assemblage properties and forming microcapsule for guest encapsulation is disclosed. Such polymer-clay nanocomposite and microcapsules are not currently available in the art.

The main objective of the present invention is to provide a nanocomposite forming microcapsule useful for guest encapsulation and process thereof.

Another objective of the present invention is to provide a process for the preparation of a polymer-clay nanocomposite exhibiting solvent assisted self-assemblage properties and forming microcapsule.

Yet another objective is to provide a process wherein a polymer-clay nanocomposite exhibiting solvent-assisted self-assemblage properties is formed by dispensing a oligosilsesquioxane-modified smectite-type clay in a vinyl polymer by in situ intercalative polymerization of a vinyl monomer.

Yet another objective is to provide a process wherein a microcapsule having a hollow-core and membrane of polymer-clay nanocomposite is formed by dissolving the nanocomposite in a suitable volatile solvent followed by casting and evaporation of the solvent.

Yet another objective is to provide a process wherein the hollow-core of the microcapsule is loaded with a guest-molecule from a suitable solvent.

### SUMMARY OF THE INVENTION

Accordingly, the present invention relates to a nanocomposite forming microcapsule useful for guest encapsulation and process thereof. This nanocomposite exhibiting self-assemblage properties and forming hollow microcapsule when dissolved in a suitable volatile solvent having dielectric constant of 2-10 followed by casting and evaporation of the solvent, the nanocomposite comprising a oligosilsesquioxane-modified clay dispensed in a vinyl polymer by in situ intercalative polymerization of a vinyl monomer. The present invention also provides a process for the preparation of polymer-clay nanocomposite, said process comprising the steps: (a) agitating a slurry of a smectite-type clay in water at 0.5-20% by weight of clay with a solution of oligosilsesquioxane derivative from a mixture of a trialkoxy aminoalkyl silane and a trialkoxy alkenyl silane in an amount of 0.2-0.8 mole of silane mixture per 100 grams of the clay, at ambient temperature for a period of 6-48 hrs, and recovering the reaction product; (b) heating at a temperature of 60-90° C. a mix of a vinyl monomer, the said reaction product in an amount of 1-20 weight percent of the monomer and a free-radical initiator in an amount of 0.5-3 weight percent of the monomer, under stirring, for sufficient time to form a solid; (c) dissolving the solid in a suitable amount of a suitable organic solvent and removing insoluble/suspended matter; (d) adding a suitable alcohol in an amount sufficient for precipitating the soluble matter; and (e) recovering the precipitate. The present invention also provides a process for preparing microcapsules from polymer-clay nanocomposite, the process comprising casting and drying on a glass plate at a temperature of 25-35° C. a solution of the nanocomposite in a suitable volatile solvent in an amount of 0.1-0.5 gram per 100 millilitre of the volatile solvent. The present invention also provides a process for producing guest-encapsulated microcapsule, the process comprising the steps: (a) preparing a solution of a guest molecule in a volatile solvent suitable for forming microcapsule, in an amount of less than 0.5 gram per 100 milliliter of the solvent; (b) dispersing nanocomposite in an amount of 0.1-0.5 gram per 100 millilitre of the solvent; and (c) casting and drying on a glass plate at a temperature of 25-35° C.

In one embodiment of the invention, a nanocomposite comprising a oligosilsesquioxane-modified clay, preferably having cation exchange capacity of at least 70 milliequivalents per 100 gram of the clay, dispensed in a vinyl polymer by in situ intercalative polymerization of a vinyl monomer selected from the group consisting of vinyl benzene and allyl benzene.

In yet another embodiment of the invention, a nanocomposite material, wherein oligosilsesquioxane-modified clay is a reaction product of a oligosilsequioxane derivative from hydrolytic poly-co-condensation of a mixture of a trialkoxy aminoalkyl silane monomer and a trialkoxy alkenyl silane monomer, and a smectite-type clay selected from the group consisting of montmorillonite, bentonite, beidellite, hectorite, saponite, sauconite and nontronite.

In yet another embodiment of the invention, a process for preparing nanocomposite material, comprising the steps of:
a. agitating a slurry of a smectite-type clay in water ranging between 0.5-20% preferably less than 10%, more preferably less than 5% by weight of clay, with a solution of oligosilsesquioxane derivative in an amount of 0.2-0.8 mole of silane mixture per 100 grams of the clay at ambient temperature ranging between 25-35° C. for a period of ranging between 6-48 hrs;
b. recovering the reaction product as obtained in step (a) by filtration followed by washing with ethanol and vacuum drying;
c. heating a mixture of vinyl monomer and reaction product as obtained in step (b) in an amount of 1-20 weight percent of the vinyl monomer, preferably 5-15% and a free-radical initiator in an amount of 0.5-3 weight percent of the vinyl monomer under nitrogen atmosphere at temperature ranging between 60-90° C. under stirring for a period of ranging between 3-5 hrs to form a solid;
d. dissolving the solid as obtained in step (c) in an organic solvent followed by cooling and removing insoluble/suspended matter by centrifugation;
e. adding an alcohol into clear solution as obtained in step (d) for precipitating the soluble matter;
f. recovering the precipitate as obtained in step (e) by filteration and drying at temperature ranging between 100-120° C. to obtain nanocomposite material.

In yet another embodiment of the invention, a process, wherein solution of a oligosilsesquioxane derivative is prepared by diluting a mixture of a trialkoxy aminoalkyl silane monomer and a trialkoxy alkenyl silane monomer at mole ratio ranging between 1:1 and 1:7, preferably 1:1 to 1:3, with alcohol-water mixture ratio in the range of 14:0.8 to 14:1.2 v/v to a solution at centration of 0.3-0.5 M of the silane mixture and aging the solution at ambient temperature for 7-10 days.

In another embodiment of the invention, a process, wherein trialkoxy aminoalkyl silane monomer is selected from the group having general formula of XSiY₃ where X is a alkyl substituted amino group comprising aminoalkyl, N-methyl substituted aminoalkyl and N,N-dimethyl substituted aminoalkyl group consisting of alkyl group having 1-5 carbon atom, preferably 1-3 carbon atom, and Y is a alkoxy group consisting of alkyl group having 1-5 carbon atom, preferably 1-3 carbon atom.

In another embodiment of the invention, a process, wherein trialkoxy alkenyl silane monomer is selected from the group having general formula of X′SiY₃ where X′ is a alkenyl group having 2-5 carbon atom, preferably 2-3 carbon atom, and Y is a alkoxy group consisting of alkyl group having 1-5 carbon atom, preferably 1-3 carbon atom.

In another embodiment of the invention, the free-radical initiator used in step (c) is selected from the group consisting of dibenzoyl peroxide, dicumyl peroxide, dilauroyl peroxide, t-butyl peroxybenzoate, and azobisisobutyronitrile.

In another embodiment of the invention, the organic solvent used in step (d) is selected from the group consisting of toluene, xylene, carbon tetrachloride, chloroform, dichloromethane, carbon disulphide, and tetra-hydro furan having dielectric constant of 2-10.

In one embodiment of the invention, the alcohol used in step (e) is selected from the group consisting of methanol, ethanol, n-propanol, iso-propanl, n-butanol, and iso-butanol.

In another embodiment of the invention, a process for preparing microcapsule from nanocomposite material, comprising the steps of:
(a) dispersing the nanocomposite material in a volatile solvent in an amount of 0.1-0.5 gram per 100 millilitre of the volatile solvent having dielectric constant between 2 and 10; and
(b) casting and drying of the solution on a glass plate at ambient temperature of 25-35° C.

In another embodiment of the invention, the volatile solvent used in step (a) is selected from the group consisting of tetrahydrofuran, carbon tetrachloride, chloroform, dichloro methane, and carbon disulphide.

In another embodiment of the invention, microcapsule is a hollow sphere having diameter of 1-10 micrometer and membrane thickness of 70-100 nanometer.

In one embodiment of the invention, a nanocomposite material, exhibiting self-assembling properties and forming microcapsule and guest encapsulate microcapsule when dissolved in a suitable volatile solvent having dielectric constant of 2-10 followed by casting and evaporation of the solvent.

In another embodiment of the invention, a guest encapsulated microcapsule is prepared by dilute solution of a guest molecule of dielectric constant below 10 in a solvent having dielectric constant between 2-10.

In another embodiment of the invention, a guest encapsulated microcapsule is prepared by addition of solution of guest molecule into the solution as obtained in step (a) in paragraph [0025] followed by step (b) in paragraph [0025].

In another embodiment of the invention, a process, wherein the guest-molecule is selected from the group consisting of drug, dye, catalyst, oil and cosmetics.

### BRIEF DESCRIPTION OF THE DRAWINGS

FIG. 1 shows the process flow-chart for preparing polymer/clay nanocomposite;

FIG. 2 shows ²⁹5i NMR spectra of (A) a oligosilsesquioxane-modified smectite clay and (B) the prestine smectite clay;

FIG. 3 shows X-ray powder diffractogram of (A) a oligosilsesquioxane-modified smectite clay and (B) the pristine smectite clay;

FIG. 4 shows (A) Scanning electron microscopy images and (B) Transmission electron microscopy images of microcapsules of polymer-clay nanocomposite; and

FIG. 5 shows Fluorescent microscope images of guest-encapsulated microcapsules (A) from solution of a fluorescent dye (Rhodamine 6G) and (B) from solution of admixture of a fatty oil and a fluorescent dye (8-anilino naphthalene sulphonic acid).

The illustrations demonstrate the chemical and structural features of a oligosilsesquioxane-modified clay, polymer-clay nanocomposite and microcapsules of this invention. The illustrations also demonstrate the formation of guest-encapsulated microcapsule of polymer-clay nanocomposite.

### DETAILED DESCRIPTION OF THE INVENTION

Disclosed herein is a polymer-clay nanocomposite exhibiting solvent-assisted self-assemblage properties and forming microcapsule, a process for preparing the polymer-clay nanocomposite, further a process for preparing microcapsule and still further a process for producing a guest-encapsulated microcapsule. From an applied perspective, the polymer-clay nanocomposite microcapsules can find guest-encapsulation applications such as micro-storage system and encapsulation/delivery of cosmetically active molecules, dyes, catalysts etc.

The polymer-clay nanocomposite of this invention is composed of a oligosilsesquioxane-modified clay dispensed in a vinyl polymer by in situ intercalative polymerization of a vinyl monomer; microcapsule is a hollow sphere having diameter of 1-10 micrometer and membrane having a thickness of 70-100 nanometer; and guest-encapsulated is a microcapsule wherein in the hollow-space of the microcapsule is loaded with a organic molecule.

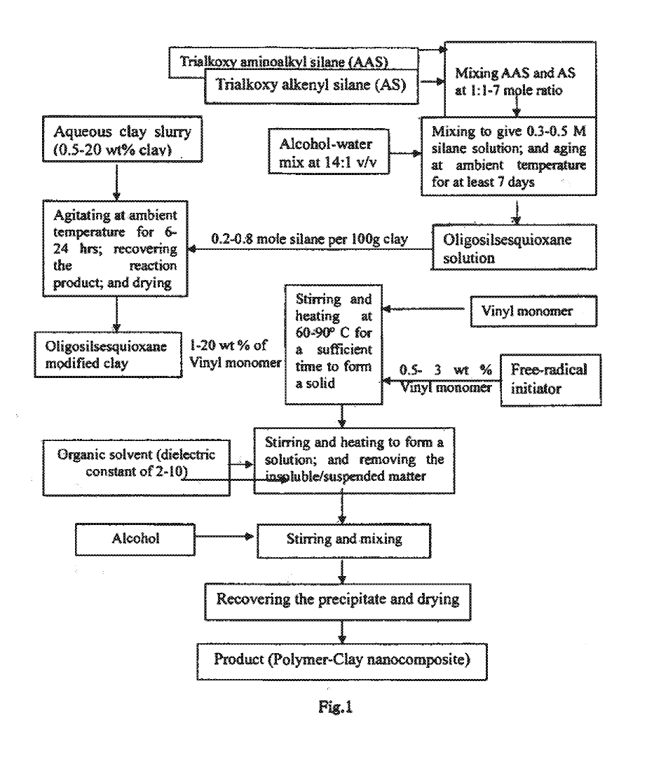

The process for the preparation of polymer-clay nanocomposite of this invention has the following steps: (a) preparing a oligosilsesquioxane-modified clay; (b) heating a dispersion of the oligosilsesquioxane-modified clay in a vinyl monomer containing a free-radical initiator to form a solid; (c) dissolving the solid in a suitable organic solvent and removing the suspended matter; and (d) recovering the soluble product. The process flow-chart is shown in FIG. 1.

The oligosilsesquioxane-modified clay is a reaction product of a smectite-type clay and a oligosesquioxane derivative. Smectite-type clays are 2:1 clays that carry a lattice charge and characteristically expand when solvated with water and alcohols. They also show cation exchange properties with inorganic and organic cations. The cation exchange capacity is expressed in terms of milliequivalent per 100 gm of the clay. The cation exchange capacity can be determined by the well-known ammonium acetate method. Commercially available smectites include montmorillonite, bentonite, beidellite, hectorite, saponite, sauconite and nontronite. Smectite-type clay with cation exchange capacity of at least 70 milliequivalent per 100 gm of the clay is used in the present invention.

The oilgosilsesquioxane derivative is a hydrolytic poly-co-condensation product of a mixture of a trialkoxy aminoalkyl silane monomer and a trialkoxy alkenyl silane monomer at mole ratio between 1:1 to 1:7. Trialkoxy silane monomers are monomers having general formula of XSiY₃ where X is a non-hydrolysable organic group, for example, an alkyl group attached to Si atom through the carbon atom and Y is a hydrolysable group, for example, alkoxy group attached to the Si atom through oxygen atom. Hydrolysis of the silane, which occurs in presence of water, under acidic or alkaline condition, leads to tri-hydroxy silane, which undergoes condensation through the hydroxyl groups by elimination of water to produce linear or cyclic or three-dimensional siloxane structure carrying the non-hydrolysable organic group attached to the Si atom. Whereas controlled hydrolytic polycondensation of XSiY₃ in alcohol-water mixture yield homo-substituted oligosilsesquioxane derivative having general formula of (XSiO_{1.5})₈, hydrolytic poly-co-condensation of a mixture of XSiY₃ and X′SiY₃ monomers having similar rates of hydrolysis usually gives hetero-substituted oligosilsesquioxane derivative having X and X′ substitutions in a ratio depending on the molar ratio of the monomer.

Oligosilsesquioxane-modified clay is prepared by following steps of: (a) preparing a slurry of clay in water at at a suitable concentration; (b) adding, while agitating the slurry, suitable amount of a solution of a oligosilsesquioxane derivative from a mixture of trialkoxy silanes; (c) continuing agitation at ambient temperature for a suitable duration; and (d) recovering the reaction product.

Slurry of clay can be prepared by vigorously agitating a mix of the clay with deionised water. Dispersion of clay can be facilitated by heating the slurry to elevated temperature, for example 60° C., and/or sonication. The slurry concentration is 0.5-20%, preferably less than 10%, more preferably less than 5% by weight of the clay.

Solution of a oligosilsesquioxane derivative is prepared by following steps of: (a) preparing a mix of a trialkoxy aminoalkyl silane monomer and a trialkoxy alkenyl silane monomer at a mole ratio between 1:1 and 1:7, preferably in the range of 1:1 and 1:3; (b) diluting the silane mixture with alcohol-water mixture of 14:1 v/v ratio to a silane solution concentration of 0.3-0.5 M, preferably 0.4-05M; and (c) aging the solution at ambient temperature for at least 7 days.

The trialkoxy aminoalkyl silane monomer is selected from the group having general formula of XSiY₃ where X is a alkyl substituted amino group comprising aminoalkyl, N-methyl substituted aminoalkyl and N,N-dimethyl substituted aminoalkyl group consisting of alkyl group having 1-5 carbon atom, preferably 1-3 carbon atom, and Y is a alkoxy group consisting of alkyl group having 1-5 carbon atom, preferably 1-3 carbon atom, and the trialkoxy alkenyl silane monomer is selected from the group having general formula of X′SiY₃ where X′ is a alkenyl group having 2-5 carbon atom, preferably 2-3 carbon atom, and Y is a alkoxy group consisting of alkyl group having 1-5 carbon atom, preferably 1-3 carbon atom.

Hydrolytic polycondensation of the silane mixture occurs without adding an external catalyst, owing to the internal catalytic activity of basic amino group of the trialkoxy aminoalkyl silane. Poly-co-condensation of the silane mixture leads to formation of hetero-substituted oligosilsesquioxane derivative containing amino and alkenyl substitutions.

Oligosilsesquioxane-modified clay is obtained by mixing the clay in water with solution of oligosilsesquioxane from the silane mixture in amount of 0.2-0.8 mole of the silane mixture per 100 grams of the clay and agitating for a duration of 6 hrs, preferably 24 hrs, more preferably for 48 hrs.

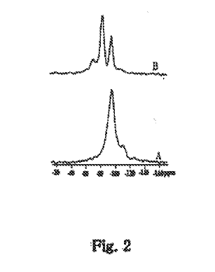

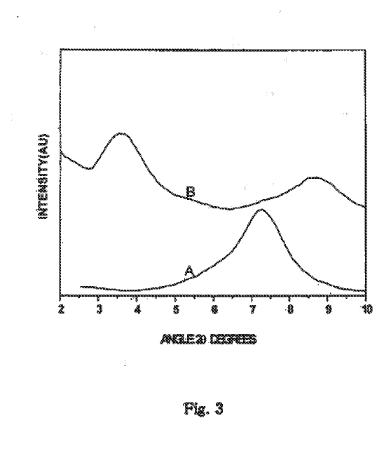

The smectite-type clay in water when agitated with oligosilsesquioxane solution gets flocculated due to organo-modification by absorption of the oligosilsesquioxane derivative into the clay galleries. The oligosilsesquioxane-modified clay is separated by centrifugation/filtration, washing with alcohol followed by drying at a temperature below 80° C. preferably under vacuum. The oligosilsesquioxane-modified clay thus prepared exhibits characteristic ²⁹Si NMR peaks due to aminoalkyl-substituted Si and vinyl-substituted Si of oligosilsisesquioxane in addition to the peak due Si of the clay. FIG. 2 shows the ²⁹Si NMR of a typical oligosilsesquioxane-modified clay in comparison with that of the unmodified clay. The oligosilsesquioxane-modified clay also shows interlayer spacing of at least 9 Å, preferably 12 Å higher than that of the un-modified clay. The interlayer spacing can be measured from the basal plane reflections in ‘X-ray powder diffractogram’. FIG. 3 shows the X-ray powder diffractogram of a typical oligosilsesquioxane-modified clay in comparison with that of the un-modified clay.

Polymer-clay nanocomposite using the oligosilsesquioxane-modified clay is prepared by in situ intercalative polymerization of a vinyl monomer. The vinyl monomer is selected from the group consisting of vinyl benzene, allyl benzene and their derivatives thereof.

The process for preparing the nanocomposite comprises the steps of: (a) dispersing suitable amount of oligosilsesquioxane-modified clay in a vinyl monomer, (b) adding suitable amount of a free radical initiator; (c) stirring and heating at a suitable temperature until the reaction mixture becomes a solid; (d) dissolving the solid in a suitable organic solvent; (e) removing the insoluble/suspended matter by filteration/centrifugation; (f) adding a suitable alcohol in amount sufficient for precipitating the soluble matter; and (h) recovering the precipitate of polymer-clay nanocomposite.

The amount of oligosilsesquioxane-modified clay in vinyl monomer is 1-20%, preferably 5-15%, more preferably 8-12% by weight of the vinyl monomer. The amount of free-radical initiator is 0.2-3.0%, preferably 1-3% by weight of the vinyl monomer.

The nanocomposite is formed by free-radical polymerization reactions involving vinyl group of the vinyl monomer and alkenyl group of the oligosilsesquioxane derivative. Polymerization is initiated by free-radical generated from free-radical initiator, such as dibenzoyl peroxide, on heating at a temperature in the range of 60-90° C. depending on the dissociation temperature of the initiator. Commercial vinyl monomer generally contains stabilizer such as hydroquinone to inhibit polymerization during storing and transportation. Stabilizer-free monomer can be prepared by washing with aqueous sodium hydroxide solution followed by distillation. Polymerization proceeds through forming a gel and then a solid. In the present invention, gelation of the reaction mixture occurs during 30-60 min. and the gel becomes a solid mass during 2-3 hrs.

The solid thus obtained is composed of 60-90% of a soluble fraction, yielding clear solutions in an organic solvent selected from the group consisting toluene, xylene, carbon tetrachloride, chloroform, carbon disulphide, and tetra-hydro furan having dielectric constant in the range of 2-10. The soluble faction exhibits solvent-assisted self-assemblage properties which can be observed by particle size measurements by the well-known “Dynamic light scattering” method using dilute solutions. The insoluble fraction, which remains suspended in the solution, can be removed by centrifugation or filtration. The soluble fraction from its solution can be recovered by precipitating by adding suitable amount of a suitable alcohol selected from the group consisting of methanol, ethanol, n-propanol, iso-propanol, n-butanol, iso-butanol and ter-butanol, which perform as non-solvents for the nanocomposite. Concentrating the solution by distilling-off the solvent can reduce the amount of alcohol needed for complete precipitation of the soluble fraction. The precipitate can be recovered by filtration/centrifugation and dried at a temperature of 80-110° C.

Microcapsules from polymer-clay nanocomposite can be prepared by the following steps: (a) preparing a solution of the nanocomposite in a suitable volatile organic solvent; and (b) casting and drying of the solution on a glass plate.

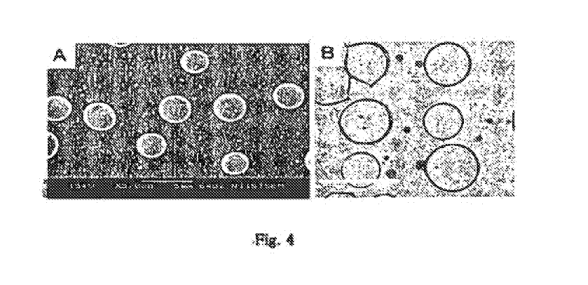

The volatile solvent for forming the solution is selected from the group consisting of carbon tetrachloride, chloroform, dichloro methane, carbon disulphide and tetra-hydro furan having dielectric constant between 2 and 10. The amount of nanocomposite in the solution is 0.1-0.5 gram per 100 millilitre of the volatile solvent. Casting and drying of the solution can be accomplished at a temperature of 25-35° C. The solution can be cast as droplets of 10-50 microlitre using suitable capillary tube. The microcapsules (hollow spheres) thus obtained can have diameter of 1-10 micrometer and membrane thickness of 70-100 nanometer. The diameter of the microcapsule can be measured by “Scanning electron microscopy” (SEM) and membrane thickness by “Transmission electron microscopy” (TEM). FIG. 4 shows the SEM and TEM images of microcapsules from polymer-clay nanocomposite of this invention.

Guest-encapsulated microcapsules can be prepared by the following steps: (a) preparing a solution of a guest molecule in a volatile solvent suitable for preparing microcapsule from the nancomposite; (b) dispersing nanocomposite in an amount of 0.1-0.5 gram per 100 millilitre of the volatile solvent; (c) casting and drying of the solution on a glass at a temperature of 25-35° C.

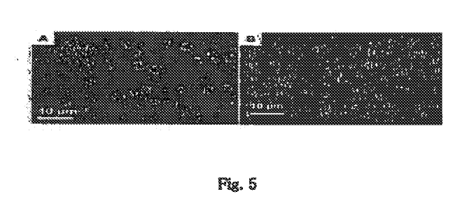

Formation of guest-encapsulated microcapsule can be checked by incorporating a fluorescent dye in the solution and viewing the microcapsule under fluorescent microscope. FIG. 5 shows the fluorescent microscopy images of guest-encapsulated microcapsules from a solution of a fluorescent dye and from a solution of admixture of an oil and a fluorescent dye.

The invention is described in detail in the following examples, which are provided by way of illustration only and should not be construed to limit the scope of the invention.

### EXAMPLE 1

Preparation of Nanocomposite.

A mix of 3.32 g (0.015 mole) of (3-aminopropyl)triethoxy silane and 4.76 g (0.025 mole) of vinyl triethoxy silane, diluted to a volume of 100 ml with ethanol-water mixture of 14:1 v/v ratio and aged at ambient temperature in a suitable closed container for a period of 10 days was added to slurry of 10 g of montmorrillonite clay in 500 ml water in a suitable vessel, agitated at ambient temperature for a period of 24 hrs, filtered and the residue was washed with ethanol, and vacuum dried at a temperature of 60° C. A mix of 5 g of the dried residue, 110 ml of vinyl benzene and 1.5 g of dibenzoyl peroxide, in a two-necked flask, under vigorous stirring and nitrogen atmosphere, was heated at 80° C. for a period of 3 hrs, added 500 ml of toluene and refluxed to form a solution, cooled to ambient temperature, centrifuged at 4000 RPM and the clear supernatant solution was concentrated to 200 ml, stirred with 100 ml of methanol, filtered and the residue was dried at temperature of 110° C.

Preparation of Micro-Capsule.

˜25 microlitre droplets of a solution of 0.2 g of the nanocomposite in 100 ml of tetrahydrofuran, in a suitable glass container, was cast on a glass plate and dried at ambient temperature.

Preparation of a Dye-Encapsulated Microcapsule.

˜25 microlitre droplets of a solution of 0.2 g of the nanocomposite and 0.05 g of 8-anilino naphthalene sulphonic acid in 100 ml of tetrahydrofuran, in a suitable glass container, was cast on a glass plate and dried at ambient temperature.

### EXAMPLE 2

Preparation of Nanocomposite.

A mix of 2.85 g (0.015 mole) of trimethoxy [3-(methylamino)propyl] silane and 6.66 g (0.045 mole) of vinyl trimethoxy silane, diluted to a volume of 130 ml with ethanol-water mixture of 14:1.2 v/v ratio and aged at ambient temperature in a suitable closed container for a period of 7 days was added to a slurry of 10 g of montmorrillonite clay in 1000 ml water, in a suitable vessel, agitated at ambient temperature for a period of 36 hrs, filtered and the residue was washed with ethanol, and vacuum dried at a temperature of 60° C. A mix of 8 g of the dried residue, 100 ml of allyl benzene and 2 g of dicumyl peroxide, in a two-necked flask, under vigorous stirring and nitrogen atmosphere, was heated at 105° C. for a period of 4 hrs, added 300 ml of toluene and refluxed to form a solution, cooled to ambient temperature, centrifuged at 4000 RPM and the clear supernatant solution was stirred with 250 ml of ethanol, filtered and the residue was dried at temperature of 110° C.

Preparation of Micro-Capsule.

˜25 microlitre droplets of a solution of 0.3 g of the nanocomposite in 100 ml of chloroform, in a suitable glass container, was cast on a glass plate and dried at ambient temperature.

Preparation of an Oil-Encapsulated Microcapsule.

˜25 microlitre droplets of a solution of 0.3 g of the nanocomposite and 0.1 g of sandalwood oil in 100 ml of chloroform, in a suitable glass container, was cast on a glass plate and dried at ambient temperature.

### EXAMPLE 3

Preparation of Nanocomposite.

A mix of 3.98 g (0.018 mole) of 3-aminopropyl triethoxy silane and 3.68 g (0.018 mole) of allyl triethoxy silane, diluted to a volume of 100 ml with ethanol-water mixture of 14:0.8 v/v ratio and aged at ambient temperature in a suitable closed container for a period of 7 days was added to a slurry of 10 g of bentonite clay in 700 ml water, in a suitable vessel, agitated at ambient temperature for a period of 18 h, filtered and the residue was washed with ethanol, and vacuum dried at a temperature of 60° C. A mix of 12 g of the dried residue, 100 ml of vinyl benzene and 3 g of dilauroyl peroxide, in a two-necked flask, under vigorous stirring and nitrogen atmosphere, was heated at 60° C. for a period of 4 hrs, added 400 ml of o-xylene and refluxed to form a solution, cooled to ambient temperature, centrifuged at 4000 RPM and the clear supernatant solution was concentrated to 200 ml, stirred with 300 ml of ethanol, filtered and the residue was dried at temperature of 100° C.

Preparation of Micro-Capsule.

˜25 microlitre droplets of a solution of 0.4 g of the nanocomposite in 100 ml of carbon disulphide, in a suitable glass container, was cast on a glass plate and dried at ambient temperature.

Preparation of a Catalyst-Encapsulated Microcapsule.

˜25 microlitre droplets of a solution of 0.4 g of the nanocomposite and 0.15 g of benzoyl peroxide in 100 ml of carbondisulphide, in a suitable glass vessel, was cast on a glass plate and dried at ambient temperature.

Advantages of the present invention:

It provides a polymer-clay nanocomposite exhibiting solvent-assisted self-assemblage properties and forming microcapsule.

It provides a process for the preparation of polymer-clay nanocomposite exhibiting solvent-assisted self-assemblage properties and forming microcapsule.

The nanocomposite microcapsule can find applications such as micro-storage system and encapsulation/delivery of cosmetically active molecules, dyes, catalysts etc.

1. A nanocomposite comprising an oligosilsesquioxane-modified clay having cation exchange capacity of at least 70 milliequivalents per 100 gram of the oligosilsesquioxane-modified clay, dispensed in a vinyl polymer by in situ intercalative polymerization of a vinyl monomer selected from the group consisting of vinyl benzene and allyl benzene.

2. A nanocomposite as claimed in claim 1, wherein the oligosilsesquioxane-modified clay is a reaction product of,
a oligosilsequioxane derivative from hydrolytic poly-co-condensation of a mixture of a trialkoxy aminoalkyl silane monomer and a trialkoxy alkenyl silane monomer, and
a smectite-type clay selected from the group consisting of montmorillonite, bentonite, beidellite, hectorite, saponite, sauconite, and nontronite.

3. A nanocomposite as claimed in claim 1 exhibiting self-assembling properties and forming microcapsule and guest encapsulate microcapsule when dissolved in a suitable volatile solvent having dielectric constant of 2-10 followed by casting and evaporation of the solvent.

4. A process for preparing a nanocomposite, said process comprising the steps of:
(a) agitating a slurry of a smectite-type clay in water ranging between 0.5-20% preferably less than 10%, more preferably less than 5% by weight of clay, with a solution of oligosilsesquioxane derivative in an amount of 0.2-0.8 mole of silane mixture per 100 grams of the clay at ambient temperature ranging between 25-35° C. for a period of ranging between 6-48 hrs;
(b) recovering the reaction product as obtained in step (a) by filtration followed by washing with ethanol and vacuum drying;
(c) heating a mixture of vinyl monomer and reaction product as obtained in step (b) in an amount of 1-20 weight percent of the vinyl monomer, preferably 5-15% and a free-radical initiator in an amount of 0.5-3 weight percent of the vinyl monomer under nitrogen atmosphere at temperature ranging between 60-90° C. under stirring for a period ranging between 3-5 hrs to form a solid;
(d) dissolving the solid as obtained in step (c) in an organic solvent followed by cooling and removing insoluble/suspended matter by centrifugation;
(e) adding an alcohol into clear solution as obtained in step (d) for precipitating the soluble matter; and
(f) recovering the precipitate as obtained in step (e) by filtration and drying at temperature ranging between 100-120° C. to obtain the nanocomposite material.

5. A process as claimed in claim 4, wherein a solution of a oligosilsesquioxane derivative in step (a) is prepared by diluting a mixture of a trialkoxy aminoalkyl silane monomer and a trialkoxy alkenyl silane monomer at mole ratio ranging between 1:1 and 1:7, and preferably between 1:1 and 1:3, with alcohol-water mixture ratio in the range of 14:0.8 to 14:1.2 v/v to a solution at concentration of 0.3-0.5 M of the silane mixture and aging the solution at ambient temperature ranging between 25-35° C. for 7-10 days.

6. A process as claimed in claim 5, wherein trialkoxy aminoalkyl silane monomer is selected from the group having a general formula of XSiY₃, where X is a alkyl substituted amino group comprising aminoalkyl, N-methyl substituted aminoalkyl and N,N-dimethyl substituted aminoalkyl group consisting of alkyl group having 1-5 carbon atom, and preferably 1-3 carbon atom, and Y is a alkoxy group consisting of alkyl group having 1-5 carbon atom, and preferably 1-3 carbon atom.

7. A process as claimed in claim 5, wherein trialkoxy alkenyl silane monomer is selected from the group having general formula of X′SiY₃ where X′ is a alkenyl group having 2-5 carbon atom, and preferably 2-3 carbon atom, and Y is a alkoxy group consisting of alkyl group having 1-5 carbon atom, and preferably 1-3 carbon atom.

8. A process as claimed in claim 4, wherein the free-radical initiator used in step (c) is selected from the group consisting of dibenzoyl peroxide, dicumyl peroxide, dilauroyl peroxide, t-butyl peroxybenzoate, and azobisisobutyronitrile.

9. A process as claimed in claim 4, wherein the organic solvent used in step (d) is selected from the group consisting of toluene, xylene, carbon tetrachloride, chloroform, dichloromethane, carbon disulphide, and tetra-hydro furan having dielectric constant of 2-10.

10. A process as claimed in claim 4, wherein the alcohol used in step (e) is selected from the group consisting of methanol, ethanol, n-propanol, iso-propanl, n-butanol, and iso-butanol.

11. A process for preparing microcapsule from nanocomposite material comprising an oligosilsesquioxane-modified clay having cation exchange capacity of at least 70 milliequivalents per 100 gram of the oligosilsesquioxane-modified clay, dispensed in a vinyl polymer by in situ intercalative polymerization of a vinyl monomer selected from the group consisting of vinyl benzene and allyl benzene, said process comprising the steps of:
(a) dispersing the nanocomposite material in a volatile solvent in an amount of 0.1-0.5 gram per 100 millilitre of the volatile solvent having dielectric constant between 2 and 10; and
(b) casting and drying of the solution on a glass plate at ambient temperature of 25-35° C. to obtain microcapsule.

12. A process as claimed in claim 11, wherein the volatile solvent used in step (a) is selected from the group consisting of tetrahydrofuran, carbon tetrachloride, chloroform, dichloro methane, and carbon disulphide.

13. A process as claimed in claim 11, wherein the microcapsule is a hollow sphere having diameter of 1-10 micrometer and membrane thickness of 70-100 nanometer.

14. A process as claimed in claim 11, wherein a guest encapsulated microcapsule is prepared by dilute solution of a guest molecule of dielectric constant below 10 in a solvent having dielectric constant between 2-10.

15. A process as claimed in claim 14, wherein the guest-molecule used in step (a) is selected from the group consisting of drug, dye, catalyst, oil and cosmetics.

16. A process as claimed in claim 11, wherein a guest encapsulated microcapsule is prepared by addition of a solution of guest molecule of dielectric constant below 10 in a solvent having dielectric constant between 2-10 into the solution as obtained in step (a) followed by step (b).
